(19) United States
(12) Patent Application Publication
(10) Pub. No.: US 2007/0250065 A1
(43) Pub. Date: Oct. 25, 2007

(54) Method and apparatus for the control of hemorrhage from major vessels

(76) Inventors: David Efron, Baltimore, MD (US); Gershon Efron, Baltimore, MD (US)

Correspondence Address: GOTTLIEB RACKMAN & REISMAN PC, 270 MADISON AVENUE, 8TH FLOOR, NEW YORK, NY, 10016-0601, US

(21) Appl. No.: 11/788,430

(22) Filed: Apr. 20, 2007

Related U.S. Application Data
(60) Provisional application No. 60/793,924, filed on Apr. 21, 2006.

(51) Int. Cl. A61F 2/30

(52) U.S. Cl. 606/75

(57) ABSTRACT

The device comprises a hemostatic staple or bar comprising a bridge and pair of pins. It is designed to be engaged with the pins straddling the edges of the inferior vena cava (“IVC”), and subsequently seated under manual pressure into the underlying bone of the vertebral column. It is intended that one bar each be applied at sites above and below the injured segment of IVC. With the devices properly engaged, the pressure held between the bar and the vertebral column mechanically approximates and fixes the anterior wall of the IVC against the posterior wall, obliterating the lumen, thereby ensuring hemostasis at the isolated (injured) segment of vein. The bars are engaged with a “pusher” or delivery rod positioned perpendicular to the IVC. The delivery rod is preferably engaged to the hemostatic bar by a clip or screw mechanism to allow rapid preparation and deployment, as well as single-handed seating of the device by the surgeon. Following the repair of the IVC, the delivery rod may be re-engaged and the bars removed, or alternatively, in an unstable patient, the bars might be left in place indefinitely.

This application claims the benefit of the filing date of U.S. Provisional Application No. 60/793,924, filed Apr. 21, 2006, entitled Method and Apparatus for the Control of Hemorrhage from Major Vessels.

### FIELD OF INVENTION

This invention relates to a method and apparatus for managing hemorrhaging. More specifically, the invention involves a method and apparatus for controlling hemorrhaging from major vessels, particularly the inferior vena cava, in the event of injury and the like.

### BACKGROUND OF INVENTION

Laceration of the inferior vena cava (“IVC”) is associated with massive hemorrhage leading to rapid exsanguination, shock and death. Expeditious control of this bleeding is vital as a life-saving maneuver. Current practice dictates manual pressure above and below the injury, and is frequently accomplished with improvised devices such as sponges fixed in clamps. Not only is this quite difficult, but it requires multiple assistants to perform, and the manual holding of such devices within the operative field greatly impairs access to and subsequent repair of the IVC injury.

In 1985, Dr. Wang Qinyao and his colleagues reported from China on the use of sterile thumbtacks to tamponade bleeding from the pre-sacral plexus of veins. See Qinyao W, Weijin S, Youren Z, Wenqing Z, Zhengrui H. New concepts in severe presacral hemorrhage during proctectomy. Arch Surg. 1985; 120:1013-1020. Anterior to the sacrum, deep in the pelvis, there is an anamtomic plexus of veins, the violation of which results in persistent and voluminous bleeding. The plexus of vessels are not controllable by vessel ligation nor by cauterization. As the remaining solution, direct pressure is applied to the injured area. By placing sterile metal thumbtacks through these vessels into the underlying bony sacrum, tamponade of the bleeding is achieved in a self-secured manner. The thumbtacks are left in place with no consequences.

For IVC injuries, thumbtacks are not an appropriate solution for a number of reasons. First and foremost, the injured IVC, where possible, should be repaired to avoid the potential sequelae of lower extremity thrombosis and edema, as well as the thrombo-embolic risk from above the injured segment. Placing thumbtacks through the IVC might achieve hemostasis, however the size of the IVC would necessitate several overlapping tacks with multiple holes through the IVC itself. If the tacks are placed directly over the injury to the vessel (as they are in the sacral plexus) this precludes repair.

### SUMMARY OF THE INVENTION

Accordingly, it is a primary object of the present invention to provide a method and apparatus effectively prevents hemorrhaging from the IVC and/or other vessels.

It is another object of the present invention to provide a method and apparatus that that stems hemorrhaging from an isolated injured segment by fixing a fastener to the underlying bone to collapse the IVC and/or other vessels.

Another object of the present invention is to provide a method and apparatus for controlling hemorrhaging that employs the concept of fixation to the underlying bone to achieve a self-affixed device that collapses the IVC and thus stems the bleeding without penetrating the IVC (and/or other vessels) itself.

It is a further object of the present invention to provide a method and apparatus for hands-free compression of the IVC and/or other vessels with control of bleeding, and allows unfettered access to the injury for potential repair.

It is still another object of the present invention to provide a method and apparatus for controlling hemorrhaging that does not significantly encumber the operative field to repair an injury to the IVC and/or other vessels.

It is yet another object of the present invention to provide a method and apparatus for controlling hemorrhaging that reduces the need for multiple assistants to participate.

Other objectives will be apparent from the description of the invention as recited herein.

The device comprises a hemostatic bar or staple, including a bridge and pair of pins at either end (modeled generally like a staple). The device is designed to be engaged with the pins straddling the edges of the IVC, and subsequently seated under manual pressure into the underlying bone of the vertebral column. It is intended that one bar or staple each be applied at sites above and below the injured segment of IVC. With the devices properly engaged, the pressure held between the bar and the vertebral column mechanically approximates and fixes the anterior wall of the IVC against the posterior wall, obliterating the lumen, thereby ensuring hemostasis at the isolated (injured) segment of vein. Following the repair of the IVC, the delivery rod may be re-engaged and the bars removed, or alternatively, in an unstable patient, the bars might be left in place indefinitely.

### BRIEF DESCRIPTION OF THE DRAWINGS

Referring to the drawings:

FIG. 1 shows the coronal view of the anterior aspect of the lumbar vertebrae from the anterior, transabdominal approach. Visualized is the overlying inferior vena cava and aorta extending to the bifurcation of the great vessels. There is shown in the mid-ascending IVC a traumatic rent. (Superiorly, visualized are the renal veins). In this figure two hemostatic staples are shown deployed in place superior and inferior to the rent. Properly placed they are deployed transversely (i.e., perpendicular) to the course of the IVC, allowing the prongs to seat into the vertebral bodies.

FIG. 2 shows the axial view of a vertebra with the overlying IVC and aorta at the level where the hemostatic staple is about to be deployed to compress the IVC. The pins or prongs are positioned extending from a bridge on either side of the IVC for insertion into the vertebra and a pusher rod is shown in dotted lines.

FIG. 3 shows the axial view of the vertebra with the overlaying IVC and aorta at the level where the hemostatic staple has been deployed (along the lines 3-3 of FIG. 1). Notable is the flattened IVC and the prongs positioned within the bony vertebra. Again, the pusher rod is in dotted lines.

FIG. 4 shows the hemostatic staple with its two pins, ready to be driven by the deploying rod in the direction of the arrow. Note the prongs straddling the IVC.

FIGS. 5 & 6 show enlarged diagrams of the hemostatic staple including a bridge and pair of pins, engaged with a pusher or delivery rod, prior to compression and at compression.

FIGS. 7 & 8 show that to secure the hemostatic staple to the deployment rod, an engaging mechanism, namely a clip-lock device, is located on the rod to engage the long sides of the hemostatic bridge, the bridge having spaced indents to engage the clip teeth. This is preferably spring-loaded to allow ease of connection to the hemostatic bar prior to placement and disconnection of the deployment rod from the deployed hemostatic bar. Other conventional clip-lock mechanisms or means can also be used for the same purpose. The clip-lock device compresses a pair of jaws, mounted for rotation on the deployment rod. The upper ends of the jaws comprise handles. The jaws are spring-biased to a closed position, whereby they grip and fit into indents in the long sides of the hemostatic bar. The jaws, by their handles, may be withdrawn from the hemostatic bar, so as to release the deployment rod from the hemostatic bar.

FIG. 9 shows the position of the rod and bar in its compression mode, crimping the IVC, prior to disengagement of the rod from the bar.

FIG. 10 shows the same apparatus, in the step of removing the pusher bar; and

FIG. 11 shows the pusher bar in its removed position.

### DETAILED DESCRIPTION OF THE INVENTION

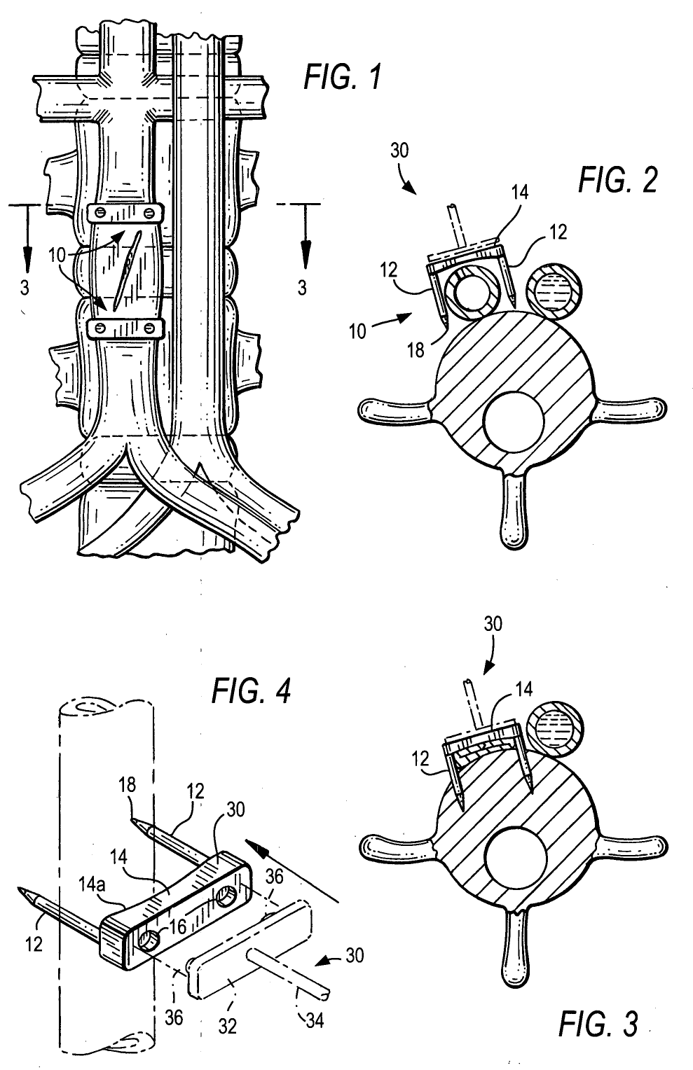

Referring to FIGS. 1 through 5, a pair of hemostatic staples 10 are shown. Each staple 10 includes a pair of parallel pins 12, which protrude from and are held rigidly by a bridge 14. The underside face of the bridge 14 is desirably concave as at 14a. The hemostatic staples 10 are driven by a pusher bar or rod 30, having a face plate 32 fixed to and driven by a stem 34.

To retain the face plate 32 and the pusher rod 30 in alignment with the staples 10 and to prevent relative motion and deter slippage, the bridge 14 has, on its upper face, a pair of apertures 16 which are in functional alignment with a pair of detents 36 on the distal face of the face plate 32, as shown especially in FIG. 4.

Preferably, the hemostatic bridge 14 is envisioned as being available in 4 different sizes. The length is preferably the only variable, wherein the bridge 14 is available in 1.5, 2.0, 2.5 and 3.0 cm lengths, with accordingly variable distance(s) between the pins (to, for example, accommodate IVCs of variable widths). The width is preferably 0.5 cm and the height is preferably 0.4 cm. The pins 12 are preferably round with a 0.15 cm diameter at the base, tapering to a point 18 over a length of 0.7 cm. They are desirably square and perpendicular to the bridge. The hemostatic bar or bridge 14 would desirably be made of titanium. It should be understood that while the foregoing comprise preferred dimensions and material for forming the hemostatic staple 10, other appropriate dimensions and materials that are known to those with skill in the art may be utilized as well or instead of the staple 10 described herein while keeping within the scope and spirit of invention.

The deployment rod 30 is envisioned as primarily a solid tool, except for the clip-lock engaging device. See FIGS. 7 & 8. The rod includes a stem 34 which is desirably 25-30 cm in length. The proximal end (i.e., the hand-held end) should widen in a comfortable grip to allow both overhand and underhand use. The distal end (i.e., the end towards the patient) would taper down to a cross bar positioned perpendicularly to the handle (that would form a “T”). The stem 34 should thus connect to a bridge or face plate 32 in its middle. The plate 32 should desirably be 0.5 cm wide, 0.5 cm thick and 2.5 cm from end to end.

An engaging mechanism is required to removably connect the pusher rod 30 to the staples 10, and one such mechanism, generally 40, is shown in FIGS. 7 and 8. Mounted on the stem 30 via a pivot rod 46 are a pair of spring-biased butterfly clips 42. The clips are biased by a pair of springs 44 to a closed position, wherein protruding members or engaging fingers 48 on the ends of the clips 42 removably slip into aligned recesses 20 in the long sides of the bridge 14.

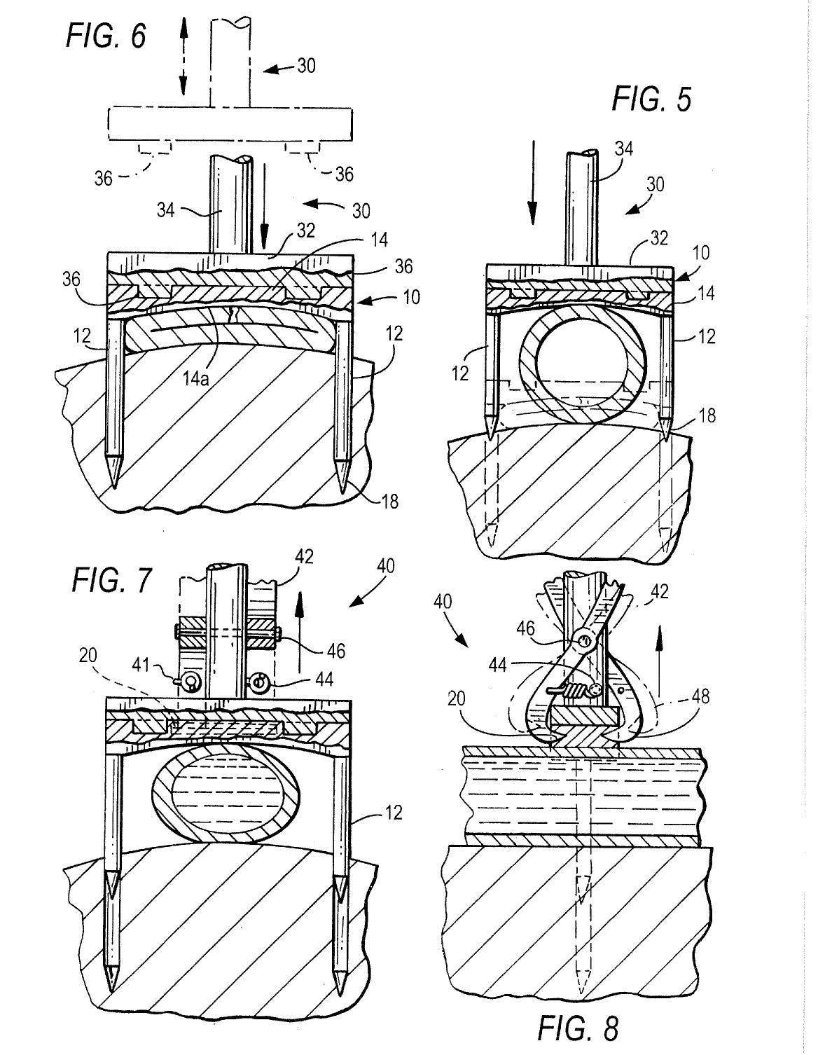

Typically, the hemostatic staple 10 is loaded onto the pusher rod 30 by the scrub technician in anticipation of its deployment. The hemostatic staple 10 is held in place via the “T” end of the pusher rod 30 by the spring loaded clips 42 that are biased to close, allowing single-handed manipulation and alignment of the device, as shown in FIGS. 7 and 8. It is important to avoid rotational motion of the staple 10 in its seating and application as this could potentially cause iatrogenic injury to the IVC as well. To prevent this, as noted, the hemostatic staple 10 is held in alignment to the “T” plate 32 of the pusher rod 30 by the detents 36 in the pusher rod 30 which are in alignment with size matched indents 20 in the hemostatic staple 10, as shown in FIGS. 5 through 8.

The size of the staple 10 used is determined by the width of the IVC. The staple 10 is applied as follows: with the medial and lateral edges of the IVC identified following modest dissection (FIG. 1), the hemostatic staple 10 is aligned with the prongs or pins 12 on either side of the IVC and seated into the vertebral column (FIGS. 3-6). The pusher rod 30 is used to apply force on the hemostatic staple 10 perpendicular to the bridge 14 thereby seating the prongs 12 into the vertebral column.

Once the repair of the IVC is achieved, the hemostatic staples 10 can be removed by reapplication of the pusher rod 30, reattaching its clips 42 and pulling them out. If the patient remains critically ill, and a damage control salvage procedure is indicated, the hemostatic staples 10 could potentially be left in place in lieu of IVC repair.

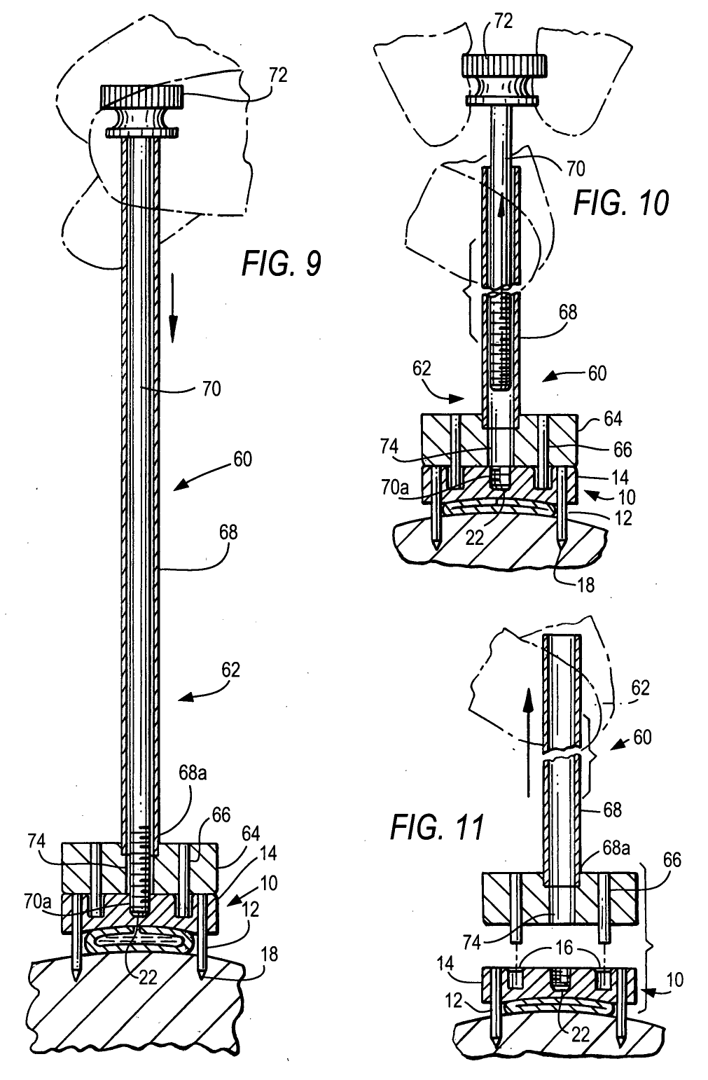

Another preferred embodiment of the stapling instrument 60 is shown in FIGS. 9-11. FIG. 9 depicts the instrument 60 combined with the staple 10 in fully assembled form and causing the staple 10 to engage and crimp the IVC. The instrument 60 engages a similar hemostatic staple 10 as discussed above which includes a pair of parallel pins 12 each ending in a sharpened point 18. The pins 12 are carried rigidly by a bridge 14 which, for alignment purposes, has a pair of apertures 16 in its upper face.

In this embodiment however, a pusher rod 62 is affixed to the hemostatic staple 10 by a screw mechanism. The pusher rod 62 engages a face plate 64 and the face plate 64 carries a pair of detents 66 which are intended to engage with and be in alignment with the two apertures 16 in the bridge 14 of the staple 10. It should also be understood that the detents and apetures on the face plate and staple, respectively, may be reversed so that the staple carries one or more of the detents, and the face plate carries one or more of the apertures.

In this embodiment, an engaging mechanism is provided to detachably (and re-attachably) attach the pusher rod 62 to a hemostatic staple 10. For this purpose, an elongated tube 68 is provided, which is fixed at its lower end 68a by conventional means to the upper portion of the face plate 64. Within the tube 68, an elongated suitably fitted screw 70 is removably housed. The screw 70 is threaded at least at its bottom end 70a and has a knurled head 72 at its top end for gripping by a user.

The face plate 64 has a through aperture 74 for the purpose of enabling the screw end 70a of the screw 70 to pass freely through it. Further, the bridge 14 of the hemostatic staple 10 of this embodiment has a threaded aperture 22 which is engaged by and adapted to receive the screw end 70a.

With this method, the hemostatic staple or bar 10 is loaded onto the pusher rod 62 by the scrub assistant and secured by engaging the screw 70 after the detents 66 and apertures 16 are properly aligned and engaged to one another. As done in connection with the embodiment discussed above, the staple 10 is affixed across and thereby crimps the IVC and the prongs 12 are seated into the vertebral bone. After being seated, the pusher rod 62 (combined with the face plate 64) is then disengaged from the staple 10 by unscrewing the mechanism, as shown in FIGS. 10 and 11. There shown is the pusher rod 62 being manually detached from the staple 10 by a user at one or more gripped or knurled surfaces, and then being withdrawn from the surgical field. The foregoing is repeated for the deployment of a second bar (and so on), as shown in FIG. 1.

In order to re-engage the rod 62 and the staple 10 for subsequent removal of the staple 10, the detents 66 on the pusher rod 62 are aligned and engaged with the apertures 16 on the face plate 14 of the staple 10. The screw 70 is then re-engaged in the threaded aperture 22 and tightened. The staple 10 is then unseated from the vertebra by pulling the combined rod 62 and staple 10 directly out, this sequence generally following FIGS. 9, 10 and 11, but in reverse.

Although the invention is described in terms of particular embodiments, it is to be understood that the embodiments are merely illustrative of an application of the principles of the invention. Numerous modifications may be made and other arrangements may be devised without departing from the spirit and scope of the invention.

1. A surgical instrument for the control of bleeding from the inferior vena cava or like major vessels adjacent to vertebrae, comprising:
a hemostatic staple, having a pair of generally parallel spaced pins and a bridge retaining said pins, said staple being dimensioned to span the vessel;
a pusher rod including a face plate and an elongated stem;
means for aligning the pusher rod with the staple so as to prevent accidental rotation therebetween; and
means for removably engaging the pusher rod to the hemostatic staple so as to enable the staple to be deployed over and to compress the vessel against a vertebra with the pins entering the vertebra and to enable disengagement and withdrawal of the pusher rod from the staple.

2. A surgical instrument as set forth in claim 1, wherein the means for aligning comprises a pair of apertures in the staple and a pair of detents carried by the pusher rod, said detents and apertures being in functional alignment.

3. A surgical instrument as set forth in claim 1 wherein the means for removably engaging comprises a pair of close-biased spring clips mounted on the pusher rod and a pair of indents in the staple, the clips engaging said indents.

4. A surgical instrument as set forth in claim 1 wherein the means for removably engaging comprises a tube fixed to the face plate, a screw held within the tube and a threaded aperture in the staple, one end of the screw removably engaging the threaded aperture.

5. A surgical instrument for the control of hemorrhaging from the inferior vena cava or like major vessels that are adjacent to bone, comprising:
a hemostatic staple having a pair of spaced pins and a bridge retaining said pins, said staple being dimensioned to span a vessel;
a pusher rod having a face plate and an elongated stem;
correspondingly matching detents and indents on the face plate and staple to prevent unintended rotation therebetween;
a protruding member carried on the rod and a receiving aperture in the staple, said protruding member being attachable and detachable from said receiving aperture as to enable the staple to be deployed over and to compress the vessel against a vertebra with the pins entering the vertebra, and to enable disengagement and withdrawal of the pusher rod from the staple.

6. The surgical instrument of claim 5, said staple having an interior surface between said pins, wherein said surface is concave.

7. The surgical instrument of claim 5, said staple having said indents and said face plate having said detents.

8. The surgical instrument of claim 5, said pusher rod further having a hollow tube and a screw, the screw being located within the tube, and said staple having a threaded aperture to receive said screw.

9. The surgical instrument of claim 8, said screw having a knurled end for manually rotating and gripping said screw.

10. The surgical instrument of claim 8, said face plate having a through aperture through which said screw passes to engage said threaded aperture in said staple.

11. The surgical instrument of claim 5, wherein said pins are parallel.

12. The surgical instrument of claim 5, wherein said pins are spaced to accommodate an inferior vena cava without puncturing it for crimping it against said vertebra.

13. The surgical instrument of claim 5, wherein said staple is fabricated from titanium.

14. A method for controlling hemorrhaging from rents in major vessels which are adjacent to a vertebra or bone, comprising:
providing a pair of hemostatic staples, each including a bridge and a pair of projecting pins;
providing a delivery rod for driving and positioning the staples;
detachably attaching the end of the delivery rod to one of said staples;
driving the staple so that it crimps the vessel and so that the pins enter the vertebra or bone on one side of the rent; and
repeating the detachably attaching and driving steps so that a staple is deployed on the other side of the rent.

15. The method of claim 14, further including the steps of:
re-attaching the delivery rod to said staple;
extracting the combined delivery rod and staple from said vertebra or bone;
repeating said re-attaching and said extracting steps for the other staple.
